(19) United States
(12) Patent Application Publication
(10) Pub. No.: US 2017/0247145 A1
(43) Pub. Date: Aug. 31, 2017

(54) A DEVICE FOR DELIVERING ODORS AND METHODS OF USING THE SAME

(71) Applicant: CORNELL UNIVERSITY, Ithaca, NY (US)

(72) Inventor: William REISACHER, New York, NY (US)

(73) Assignee: CORNELL UNIVERSITY, Ithaca, NY (US)

(21) Appl. No.: 15/514,177

(22) PCT Filed: Sep. 24, 2015
(86) PCT No.: PCT/US2015/051912

§ 371 Date: Mar. 24, 2017

Related U.S. Application Data
(60) Provisional application No. 62/054,654, filed on Sep. 24, 2014.

(51) Int. Cl. B65D 25/04; B65D 43/14; A61Q 13/00; A61K 8/36; A61K 8/37; A61K 8/35; A61K 8/46; A61K 8/49; A61K 8/33; A61K 8/40; A61K 8/34; A61B 5/00; A61K 8/92

(52) U.S. Cl. CPC B65D 25/04; A61B 5/4011; B65D 43/14; A61Q 13/00; A61K 8/36; A61K 8/37; A61K 8/922; A61K 8/46; A61K 8/49; A61K 8/4986; A61K 8/33; A61K 8/498; A61K 8/4973; A61K 8/40; A61K 8/4926; A61K 8/494; A61K 8/347; A61K 8/35; A61K 8/34

(57) ABSTRACT

Disclosed herein are kits, devices and methods for promoting, supporting and enhancing olfactory function. The kits of the present disclosure relate to devices for stimulating olfactory function that include odorous pods selected from a group of odors or more than one group of odors, whereby each odorous pod is located in a compartment of a receptacle that can be used to promote, support and enhance an individual's olfactory function. The kits of the present disclosure can also include instructional methods for using such devices to promote, support and enhance olfactory function, diagnosing or evaluating olfactory function in a subject or treating and improving olfactory function in a subject.

### CROSS REFERENCE TO RELATED APPLICATION

This application claims the benefit of U.S. Provisional Application No. 62/054,654, filed on Sep. 24, 2014, the entire contents of which are incorporated herein by reference.

### FIELD OF THE INVENTION

This disclosure generally relates to kits, devices and methods for promoting, supporting and enhancing olfactory function. More specifically, the present disclosure relates to kits for stimulating olfactory function that include odorous pods selected from a group of odors or more than one group of odors, whereby each odorous pod is located in a compartment of a receptacle. The kits of the present disclosure can also include instructional methods for using such devices to promote, support and enhance olfactory function or to diagnose or treat olfactory dysfunction.

### BACKGROUND OF THE DISCLOSURE

Olfactory function decreases with increasing age. Approximately 25% of adults over 43 years of age and one third of people over 53 years of age report impaired olfactory function. Impairment of olfactory function (i.e., olfactory dysfunction) has been linked to specific disease states including malnutrition, food poisoning, anxiety, depression, diabetes, renal disease, epilepsy and premature death. Olfactory dysfunction is also related to neurodegenerative disorders, and has been an identifying factor for the onset of Parkinson's disease. Olfactory dysfunction negatively impacts those afflicted. For example, impairment of an individual's ability to smell distorts taste and decreases a subject's quality of life, which decreases appetite and can lead to malnutrition. Moreover, individuals with decreased olfactory function are less likely to notice gas leaks, and thus are at risk for gas poisoning and explosion-related trauma.

Treatment of olfactory dysfunction is extremely limited. Depending on the cause of the disorder, treatments vary both in type and effectiveness. For example, certain olfactory disorders are triggered by upper respiratory infections, and can resolve spontaneously; while tumors and sinus disease associated olfactory disorders require diagnosis and subsequent treatment (e.g., antibiotics, steroids and/or surgery). However, if the olfactory disorder is unrelated to an underlying disease, patients are left with no options except to wait for spontaneous resolution or to try unsubstantiated alternative approaches such as acupuncture and supplements (e.g., vitamins).

Generally, olfactory training involves repeatedly exposing a subject to one or more odorants over a period of time in order to increase the subject's olfactory sensitivity. Many studies have been carried out in order to further elucidate a method or combination of odors capable of improving or restoring olfactory function. In one study, subjects without reported olfactory dysfunction, were repeatedly exposed to androstenone (i.e., a fragrance). This exposure resulted in a decreased detection threshold such that the subjects were better at detecting the androstenone odorant. See Wang, L., et al. (2004) Evidence for Peripheral Plasticity in Human Odour Response. Journal of Physiology 554(Pt 1):236-44. Another study on individuals without olfactory dysfunction showed that subjects' displayed improved olfactory function after repeated exposure to a set of odorants. See Dalton, P., et al., (2002) Gender-specific Induction of Enhanced Sensitivity to Odors. Nature Neuroscience 5(3):199-200. In other studies, repeatedly exposed a subject exhibiting olfactory loss to four strong odors revealed that training subjects with certain odors increased olfactory sensitivity for the specific odorants and in general, while untrained subjects exhibited no change in olfactory function. See Hummel, T., et al., (2009) Effects of Olfactory Training in Patients with Olfactory Loss. The Laryngoscope 119(3):496-9. Other studies concluded that training with high concentrations of odorants was beneficial in improving olfactory function. See Damm, M., et al., (2014) Olfactory Training is Helpful in Postinfectious Olfactory Loss: A Randomized, Controlled, Multicenter Study. The Laryngoscope 124(4):826-31. Other research has focused on the effects of olfactory training as a preventative treatment for olfactory deterioration in the elderly. However, elderly subjects did not exhibit a significant improvement in olfactory function as a result of training, but did show improved olfactory test scores. Schriever, et al., (2014) Preventing Olfactory Deterioration: Olfactory Training may be of Help in Older People. J Am Geriatr Soc. 62(2):384-6. As a result, a training interval of greater than three months was suggested as necessary to overcome the ongoing deterioration and decreased regenerative ability in elderly patients. Seventy subjects with Parkinson's disease (PD) related olfactory loss were examined before and after olfactory training using the “Sniffin' Sticks” test, in addition to threshold tests for odors used during the training process. Trained PD patients experienced a significant increase in their olfactory function, while olfactory function was unchanged in PD patients who did undergo olfactory training. See Haehner A, et al., (2013) Olfactory training in patients with Parkinson's disease. PLoS One. 8(4):e61680.

The foregoing studies reveal a plethora of non-uniform methods for analyzing olfactory function. However, a single, improved kit and method to promote, support, or enhance olfactory function has not yet been developed.

### SUMMARY OF THE DISCLOSURE

The present disclosure includes new kits for presenting odorous compounds (i.e., odorous chemicals), such that the end user of the kit will enhance or further develop olfactory function. One aspect of the present disclosure provides kits for olfactory stimulation including at least one compartment and at least one odorous pod. In some embodiments, the kits of the present disclosure can include instructions for use, whereby the instructions enable a user to promote, support, or enhance olfactory function using the kit. In some embodiments, the kits of the present disclosure include instructions that communicate how to treat hyposmia or anosmia using the other elements of the kit. In other embodiments of the present disclosure, kits for olfactory stimulation include a receptacle having at least one compartment in which at least one odorous pod can be placed. In specific embodiments the receptacle may have one, two, three, four, five, six, seven, eight, nine, ten, eleven, or twelve compartments. The odorous pods of the present disclosure include, either alone or in combination, odorants selected from four groups of odorants. In other embodiments, the kits of the present disclosure include a receptacle having at least four compartments, whereby each compartment includes an odorous pod or more than one odorous pod, and at least one of the odorous pods located in each one of the compartments contains an odorant selected from each of the four groups of odorants.

Another aspect of the present disclosure provides methods to promote, support, or enhance olfactory function of a subject with olfactory dysfunction using a kit of present disclosure. In one embodiment of the present disclosure, such methods can be used to treat hyposmia or anosmia in a subject in need thereof. In certain embodiments, methods of present disclosure include providing an odorant or series of odorants to be exposed and inhaled by a subject in need thereof. In other embodiments, the exposure and inhalation process of the present methods can be repeated more than once. In yet another embodiment, the exposure and inhalation process can be repeated between 1 and 3 times per day, or more. In other embodiments, the exposure and inhalation process can be repeated daily for at least one week, for at least one month, or for between 1 month and 60 months, inclusive.

### BRIEF DESCRIPTION OF THE DRAWINGS

FIG. 1 shows an embodiment of a receptacle 100 component of a kit of the present disclosure, which includes six compartments 3. Here, the first compartment is shown in an “open” configuration whereby the cover 2 has been removed from the first compartment. The remaining compartments 3 are shown in a closed configuration (e.g., sealed) configuration 4. Each compartment 3 contains an odorous pod 5 (shading) that, when released, emits an odorant 6 to be inhaled. In this embodiment, the corners of the receptacle are curved 1.

FIG. 2 shows a specific embodiment of a receptacle 200 component of a kit of the present disclosure that includes four compartments 3. Here, the first compartment 3a and the second compartment 3b are shown in an “open” configuration whereby the cover 2 has been removed from the first 3a compartment and the second compartment 3b. The remaining compartments 3c, 3d are shown in a closed configuration (e.g., sealed) configuration 4. Each compartment 3 contains an odorous pod 5 (shading) that, when released, emits an odorant 6a, 6b to a subject to be inhaled in accordance with the methods of the present disclosure.

Table 1 is a list of odorants classified by group. Each of the 4 groups of odorants are further classified into categories, and non-limiting examples of odorant chemicals associated with category of odorants are provided.

### DETAILED DESCRIPTION

Generally, it has been recognized herein that olfactory stimulation through exposure and inhalation of specific odorants promotes, supports and/or enhances olfactory function. Accordingly, the present disclosure provides a kit that includes at a receptacle composed of compartments that include at least one odorous pod selected from a group of odors. The kit may include instructions for using the same. Methods for stimulating olfactory function (e.g., treating anosmia or hyposmia in a subject), and for promoting, supporting and/or enhancing olfactory function are also provided.

References in the specification to “one embodiment”, “an embodiment”, “specific embodiment”, or a “preferred embodiment”, indicate that the embodiment described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. For purposes of the description hereinafter, the terms “upper”, “lower”, “right”, “left”, “vertical”, “horizontal”, “top”, “bottom”, “above” “below” “beneath” and derivatives thereof shall relate to the invention, as it is oriented in the drawings . The terms “overlying”, “atop”, “positioned on” or “positioned atop” means that a first element, is present on a second element, wherein intervening elements interface between the first element and the second element. The terms “contact”, “affixed to” or “attached to” mean that a first element and a second element are connected at an interface of the two elements.

### Definitions

The term “receptacle” as used herein means a base member in which a plurality of recessed compartments is formed.

The term “compartment” as used in the present disclosure means a recessed storage space, chamber or cavity located within a receptacle having a bottom surface that is located below a topmost horizontal surface of the receptacle.

An “odorous pod” as used herein means an encapsulated solution or formulation composed of at least one odorous chemical (i.e., odorant).

The term “odorant”, “odorant chemical” or “fragrance” as used herein shall mean a chemical entity, compound, molecule, ion or combination thereof that is capable of emitting a distinct scent or fragrance, which can be detected by the olfactory system.

The term “formulation” as used herein means a mixture of materials, e.g., active ingredients such as chemical odorants, and pharmaceutical carriers, e.g., excipients, bulking agents, and fillers, combined to modulate the scent released by certain odorous pods. For example, a formulation of odorant chemicals may be a pharmaceutically acceptable formulation including, but not limited to, at least one active ingredient (i.e., odorant chemical) and at least one pharmaceutical carrier (e.g., excipient, filler and bulking agent). The phrase “pharmaceutically acceptable” is employed herein to refer to those compounds, chemicals, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the odorant. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations. Wetting agents, gelling agents, emulsifiers and lubricants, such as sodium lauryl sulfate and magnesium stearate, as well as coloring agents, release agents, antimicrobial and other preservative agents, and antioxidants can also be present in such formulations. A formulation of the present disclosure can also include a gel, a gelling agent, an odorant chemical or a plurality of odorant chemicals, a surfactant and a cosolvent.

The phrase “subject” or “subject in need thereof” as used herein refers to any mammalian subject in need of a treatment, particularly an individual subjects exhibiting olfactory dysfunction, including hyposmia or anosmia. The methods of the current disclosure can be practiced on any human subject that has olfactory dysfunction.

### Olfactory Stimulating Kits.

One aspect of the present disclosure provides kits for olfactory stimulation including at least one compartment; and at least one odorous pod. In certain embodiments the receptacle of the present disclosure can be elongated and confer a rectangular or oval shape. In other embodiments the receptacle is square, or circular. However, the receptacle can form any shape known by one of ordinary skill in the art. In certain embodiments the receptacle can include a plurality of removable covers (e.g., lids), whereby each removable cover corresponds to a compartment located within the receptacle. In some embodiments each cover is affixed to a top surface of the receptacle by a hinge mechanism located at one end of each cover. In one embodiment a cover can be secured over each compartment by locking into place, magnetism, use of an adhesive or other mechanical means. A receptacle of the present disclosure can be composed of a polymer (e.g., plastic), glass, metal, wood or other malleable material capable of being molded into a desired shape. In certain embodiments the material that comprises the receptacle is microwave or dishwasher safe (e.g., high density polyethylene, and glass).

In certain embodiments of the present disclosure, kits for olfactory stimulation include at least one receptacle having at least one compartment in which at least one odorous pod can be placed. In other embodiments a kit of the present disclosure contains more than one receptacle. In a preferred embodiment a receptacle contains a plurality of compartments. In specific embodiments the receptacle may have one, two, three, four, five, six, seven, eight, nine, ten, eleven, or twelve compartments. In one embodiment a receptacle of the present disclosure includes up to twenty-four compartments. In some embodiments, each compartment can have a different shape, depth, color, and/or may be labeled. In other embodiments each compartment is of uniform size, shape, depth, and/or color. In certain embodiments each compartment is recessed to a depth of, for example, about one-half inch below the topmost surface of the receptacle, although lesser or greater depths are acceptable. Generally, each compartment is configured to house an odorous pod of the present disclosure.

The odorous pods of the present disclosure include, either alone or in combination, odorants selected from four groups of odorants. In certain embodiments an encapsulated solution containing an odorous chemical can be formulated in an odorous pod. Odorants may be formulated to produce scents that vary in strength by varying the concentration of odorous chemical or the ease with which the odorant is released or activated. Accordingly, odorous pods may be labeled as “extra strength”, “strong”, “mild” or the like. In certain embodiments of the present disclosure a receptacle, compartment, odorous pod and/or combinations thereof include a signaling element that informs the user that a certain odorant has been exhausted (i.e., is no longer sufficient to promote, support and/or enhance olfactory function of the user). Non-limiting examples of signaling elements include visual aids, such as dyes, stains, or colorized chemical odorants, which enable a user to visually detect the absence of chemical odorants (e.g., loss of color) in an odorous pod or compartment when the odorant has been exhausted.

In specific embodiments the formulation can be liquid, gelatinous or solid in nature. In another example, a series of odorant pods may be provided in a kit, whereby each odorant pod in the series of odorant pods contains a formulation of odorant chemicals in which the concentration of odorant(s) differs from the concentration of odorant(s) in the formulation present in other odorant pods in the series. By way of example, the concentration across a plurality of odorous pods decrease sequentially, whereby the first odorous pod in a series of pods contains, for example, about 10 percent by weight odorant chemical, a second odorous pod in the serious of pods contains, for example, from 9 to 7 percent by weight odorant chemical, a third odorous pod in the serious of pods contains, for example, from 6 to 3 percent by weight odorant chemical, and a fourth odorous pod in the serious of pods contains, for example, from 3 to 1 percent by weight odorant chemical. Greater or lesser amounts of odorant chemicals are also contemplated.

In one embodiment the solution that contains at least one odorous chemical is encapsulated in a bead, a packet, a rigid or semi-rigid container or other malleable material capable of entrapping a solution without leaking or evaporation therefrom, and fitting snugly in a compartment. In certain embodiments an odorous pod of the present disclosure may have an uppermost surface composed of a permeable membrane that permits dispersion of the odorant into the surrounding environment when exposed to air. Non-limiting examples of materials that can be used to form a high-surface area membrane of the present disclosure include, for example, polyethylene, polytetrafluoroethylene, polypropylene, or polyethyleneterephthalate.

An odorous pod of the present disclosure releases a scent produced by the odorous chemical or chemicals provided in each odorous pod. An odor can be released continuously or only upon activation by a user of the kit. Activation can occur, for example, through application of physical force, heat, or by introduction of a chemical. In one embodiment, an odorous pod may be activated by heating, for example, with a microwave, hair dryer, or a heating element incorporated in the receptacle or compartment. In another embodiment, an odorous pod is activated by applying physical force, such as, removing a cover layer adhering to a surface of an odorous pod such that removing the cover exposes the odorant to the external environment. Specific examples of activation of an odorous pod by physical force include, but are not limited to, removing a cover from a compartment, rupturing or releasing an encapsulated odorant chemical from beads, or removing a barrier layer separating two components of a formulation that results in mixing of chemical odorants and release of a specific scent to the user. Odorants can also be formulated as a coating (e.g., adhesive) on or between layers of sheets, whereby removal of one sheet of the layers of sheets results in the release and activation of an odorant to the user.

In an embodiment of the present disclosure, the odorous pod includes a wick made of porous polymeric material which is in contact with a formulation of an odorous chemical. The wick, through capillary action, provides a means for transporting the odorant from within the odorous pod to the outside environment. In certain embodiments the wick is contained entirely within the odorous pod and is exposed to the air once the odorous pod is activated. In another embodiment of the present disclosure a wick may transmit the odorant from within the odorous pod to a high-surface area membrane affixed to a topmost surface of the odorous pod for dispersion of the odorant into the air. Non-limiting examples of materials that can be used to form a wick of the present disclosure include polyethylene, polytetrafluoroethylene, polypropylene, or polyethyleneterephthalate.

Odorants of the present disclosure can be classified into four groups of odors, as set forth in Table 1, below. Specifically, a first group of odorants includes odorant chemicals from five categories of aromas including: acid, alliaceous, balsamic, marine, and spicy. A second group of odorants includes odorant chemicals from five different categories of aromas including: burned, aromatic, empyreumatic, herbal and woody. A third group of odorants includes odorant chemicals from eight different categories of aromas including: caprylic, aldehydic, ambrosial, hircine, nauseous, resinous, putrid, and repulsive. A fourth group of odorants includes odorant chemicals from four different categories of aromas including: fragrant, ethereal, fruity and sweet fragrances.

Each category of odorants is further delineated by specific types of odorant chemicals capable of emitting a scent corresponding to the particular category of odorant. Table 1 of the present disclosure provides a non-exhaustive list of odorant chemicals associated with each category of odorant, which further define each of the four groups of odorants.

In one embodiment of the present disclosure, at least four odorous pods are provided in a kit, whereby each pod contains an odorous chemical that emits a scent from one of the four groups of odors. In another embodiment, each pod in a group of pods contains a different odorant. In yet another embodiment, at least one pod in a group of pods contains at least one odorant from group 1, 2, 3 or 4, and the odorant(s) in each pod is selected from a different group than the odorant(s) in the other pods. In a preferred embodiment, each kit will have at least one odorous pod from each of the four groups of odorants. For example, a first odorous pod can contain and odorous chemical or more than one odorous chemical from group 1, as set forth in Table 1, while a second odorous pod contains an odorous chemical or more than one odorous chemical from group 2, 3 or 4 of Table 1. By way of example, if the second odorous pod contains an odorous chemical or chemicals from group 2, a third odorous pod will include an odorous chemical or more than one odorous chemical from group 3 or 4, only. Here, if the third odorous pod includes an odorous chemical or more than one odorous chemical from group 3, a fourth odorous pod of the group of odorous pods will include an odorous chemical or more than one odorous chemical from group 4. Other combinations and orders are also contemplated.

In one embodiment of the present disclosure, a kit includes four odorous pods, whereby each pod produces only one of the four odors from the following categories of odorants: fragrant, acid, burned, and caprylic. See Crocker, E. C. and Henderson, L. F. (1927) Analysis and classification of odors. American Perfumer and Essential Oil Review, 22, 325-327.

In another embodiment of the present disclosure, a kit includes six odorous pods, whereby each pod produces only one of the six odors from the following categories of odorants: sweet, putrid, fruity, spicy, burned, and resinous. See Henning, H. (1916) Der Geruch. Leipzig, Germany: Johann Ambrosius Barth; see, also Carrasco, M. and Ridout, J B. (1993) Olfactory perception and olfactory imagery: a multidimensional analysis. J Exp Psychol Hum Percept Perform. 19(2):287-301.

In an embodiment, a kit of the present disclosure includes seven odorous pods, whereby each pod produces only one of the seven odors selected from the following categories of odorants: aromatic, fragrant, ambrosial, alliaceous, hircine, repulsive, and nauseous. See Gilbert, A. (2008) What the Nose Knows. Crown; First Edition. In another embodiment a kit of the present disclosure includes seven odorous pods, whereby each pod produces only one of the seven odors selected from the following categories of odorants: ethereal, camphoraceous, musky, floral, spicy, sweet, and putrid. See Amoore J E, (1963) Stereochemical theory of olfaction. Nature 198 (4877): 271-2.

In a specific embodiment, a kit of the present disclosure includes nine odorous pods, each pod producing one of the nine odors according to the Zwaardemaker odor classification system. For example, such odors can be selected from the following categories of odorants ethereal (e.g., ether or beeswax), aromatic (e.g., spice or camphor), fragrant (e.g., lavender or rose petals), ambrosiac (e.g., amber or musk), alliaceous (e.g., garlic or onion), empyreumatic (e.g., roasted coffee or tobacco smoke), hircine (e.g., strong cheese or rancid food), foul (e.g., bedbugs or coriander flower), and nauseous (e.g. feces or rotten eggs). See A Dictionary of Psychology, third edition. Zwaardemaker smell system. Oxford University Press; see, also Hendrik Zwaardemaker. (1895) Die physiologie des geruchs. W. Engelmann.

In yet another embodiment, a kit of the present disclosure includes twelve odorous pods, each pod producing one of the twelve odors according to Ann Noble's “Aroma Wheel” wine odor classification system. More specifically odors for use in the present embodiment can be selected from the following categories: nauseous (e.g., sulfur and petroleum); pungent (e.g., alcohol); aldehydic (e.g., acetaldehyde); acid (e.g., yeast and lactic acid); spicy (e.g., licorice and anise); fruity (e.g., blackcurrant and apricot); herbal (e.g., eucalyptus and artichoke); aromatic (e.g., walnut and hazelnut); sweet (e.g., butterscotch and molasses); woody (e.g., oak, vanilla and coffee); and ambrosial (e.g., mushroom and mildew).

In yet another embodiment of the present disclosure, a kit can include fourteen odorous pods, each pod producing one of the fourteen odors in the “Fragrance Wheel” designed by Michael Edwards. Odors for use in the present embodiment can be selected from the following categories: floral; soft floral; floral oriental; soft oriental; oriental; woody oriental; woody; mossy woods; dry woods; aromatic; citrus; fruity; green; and water. See Edwards, M. (2011) Fragrances of the world 2011, Michael Edwards & Co.

Table 1, below provides an exemplary list of odorants classified by group, categories of each group and odorant chemicals within each category. However, the groups provided herein are not limited to only those categories shown in Table 1, nor are the categories limited to the example odorous chemical compounds provided. In addition, in some embodiments, classes or odorant groups may be defined. For example, a class, Class A of odorants includes Group 1 and Group 2, and another class, Class B includes Group 3 and Group 4. In other embodiments a class, Class C, includes Group 1 and Group 3 and another class, Class D, includes Group 2 and Group 4. In other embodiments a class, Class E, includes Group 1 and Group 4 and another class, Class F, includes Group 2 and Group 3. Other classifications are known in the art or may be designed to further define a specific set of odors for use in kits and methods of the present disclosure. For example, odorants are described in handbooks such as: Steffen Arctander, (1994) Perfume and Flavor Materials of Natural Origin. Allured Pub Corp; First Edition, and Steffen Arctander (1969) Perfume and Flavor Chemicals Aroma Chemicals Montclair N.J. USA; as well as lists and databases such as the International Fragrance Association (IFRA) list of ingredients or the Research Institute for Fragrance Materials (RIFM) database.

TABLE 1
GROUP 1 ODORS

| Categories of Group 1 Odors | Exemplary Odorous Chemicals |
|---|---|
| Acid | 2-ethyl butyric acid |
| | 2-methyl butyric acid |
| | 2-methyl hexanoic acid |
| | Phenyl pyruvic acid |
| Alliaceous (onion, garlic, caoutchouc, | Allyl propionate |
| acetylene, chlorine, iodine) | Asafetida oil |
| | Dibutyl sulfide |
| | Tropical thiazole |
| | 2-acetyl thiophene |
| Balsamic | Vanillin |
| | Coumarin |
| | Piperonal |
| Marine (ozone) | Calone |
| | Dodecane nitrile |
| | Ultrazur |

GROUP 2 ODORS

| Categories of Group 2 Odors | Exemplary Odorous Chemicals |
|---|---|
| Burned also known as tarry | 3-acetyl pyridine |
| | Isopropenyl pyrazine |
| | 4-isopropyl phenol |
| | 2,4-xylenol |
| Aromatic (camphor, spices, clove, | Butyl salicylate |
| aniseed, lemon, almond, turpentine) | Cinnamon leaf oil |
| | Clover nitrile |
| | Cyclademol |
| | Geranic oxide |
| Empyreumatic (coffee, baked bread, | 2-acetyl-6-methyl pyrazine |
| tobacco smoke, kerosene, asphalt, | Coffee dione |
| carbolic acid) | 1-hydroxy-2-butanone |
| | 8-nonen-2-one |
| | Tobacco flower absolute |
| | Para-cresyl isovalerate |
| Herbal | Cis-3-hexenol |
| | Menthol |
| | Isomenthone |
| Woody | Patchouli alcohol |
| | Beta-Santalol |
| | Bacdanol |

GROUP 3 ODORS

| Categories of Group 3 Odors | Exemplary Odorous Chemicals |
|---|---|
| Caprylic | Octanoic acid aka Caprylic acid |
| | humulone, isohumulone and |
| | humulene |
| Aldehydic | Aldehyde c-8 |
| | Aldehyde c-10 |
| | Aldehyde c-12 |
| Ambrosial (amber, musk) | (Z)-isoambrettolide |
| | Exaltone |
| | Serenolide |
| | Traseolide |
| | Ambrinol |
| | Formoxymethyl isolongifolene |
| | Civettone |
| | Muscone |
| | Muskalactone |
| Hircine (valerian, old cheese, sheepskin, | Bornyl isovalerate |
| perspiration) | Valerian rhizome absolute |
| | Spikenard oil |
| | 3-methyl-3-penten-2-one |
| | Neryl isovalerate |
| Nauseous (carrion) | Dimethyl sulfide |
| Resinous aka Paint-like | Formaldehyde |
| | Methyl hexyl ether |
| | Waxy lactone |
| Putrid | Ethyl isobutyrate |
| | Ethyl vinyl ketone |
| | 2-pentenal |
| | Isovaleric acid |
| Repulsive (also known as Foul, e.g., | (S)-gamma-nonalactone |
| opium and bedbugs) | Menglytate |
| | 2-pyridyl carbinol |

GROUP 4 ODORS

| Categories of Group 4 Odors | Exemplary Odorous Chemicals |
|---|---|
| Fragrant (lily) | Cirtonellyl ethoxalate |
| | Muguet butanol |
| | Hydroxycitronellal nitrile |
| | Freesia acetate |
| | Hydroxycitronellol |
| Ethereal (ether, apples, wine, beeswax) | Acetaldehyde dimethyl acetal |
| | Isoamyl acetoacetate |
| | Alpha-amyl cinnamaldehyde |
| | diethyl acetal |
| | Isopropenyl acetate |
| | Ethyl acetate |
| Fruity | Ally heptonate |
| | Dicyclopentadiene propionate |
| | Nonanal propylene glycol acetal |
| | Raspberry dicarboxylate |
| | Styralyl butyrate |
| | Aldehyde c-14 |
| | Ethyl butyrate |
| | Fruitate |
| Sweet (also known as “floral”) | Allyl hexanoate |
| | Isoamyl phenyl acetate |
| | Isobutyl isovalerate |
| | Caramel |
| | Cyclotene hydrate |
| | Ethyl benzoate |
| | Ethyl cyclopentenolone |
| | Ethyl furaneol |
| | Phenyl acetate family |
| | Musk cyclopentenyl propionate |
| | Strawberry furanone family |
| | Vanilla family |
| | Phenyl ethyl alcohol |
| | Hedione |
| | Beta-ionone |

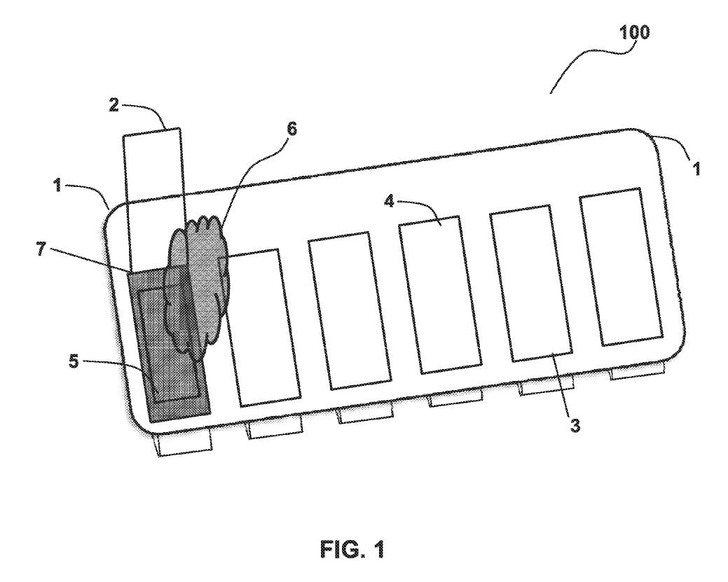

One embodiment of the kit of the present disclosure is shown in FIG. 1. In the embodiment shown in FIG. 1, a single receptacle 100 is depicted, which includes six compartments 3. However, the kit can include more than one receptacle. For example and within the limits described here, the kit can include two, three, four, five or six receptacles, each of which includes at least one compartment. Other variations within the limits provided here are also possible.

In the specific embodiment shown in FIG. 1, each compartment has a removable cover 2 whereby each removable cover 2 corresponds to a compartment 3 located within the receptacle 100. Each cover is affixed to a top surface of the receptacle 100 by a hinge mechanism 7 located at one end of each cover 2. In other embodiments (not shown), the cover may be affixed to the receptacle 100 by snapping the cover 2 into place, such that the cover 2 is removably secured to a topmost surface of each compartment 3. FIG. 1 also shows a first compartment 3 of the receptacle 100 in the open position (e.g., cover open), which exposes the odorous pod 5 to the environment and emits a scent 6 to be inhaled by a subject.

In one embodiment, a kit of the present disclosure includes instructions for use. For example, the instructions provided within a kit enable a user to promote, support, or enhance olfactory function using the kit. In another non-limiting example, the kits of the present disclosure include instructions that communicate how to treat hyposmia or anosmia using the other elements of the kit.

### Methods to Promote, Support, or Enhance Olfactory Function.

The current disclosure also provides methods to evaluate olfactory function; to diagnose hyposmia or anosmia; to promote, support, and/or enhance olfactory function; and to treat hyposmia or anosmia in a subject in need thereof. Hence, another aspect of the present disclosure provides methods to promote, support, or enhance olfactory function of a subject with olfactory dysfunction using a kit of present disclosure. In one embodiment such methods can be used to treat hyposmia or anosmia in a subject in need thereof.

In certain embodiments, the methods of present disclosure include providing a receptacle that includes at least one compartment for housing an odorous pod including an odorant or series of odorants to be inhaled by a subject. In a specific embodiment, a method of the present disclosure provides a subject with a receptacle that includes at least four compartments for housing an odorous pod. The subject is then provided with a plurality of odorous pods, whereby the odorant in each odorous pod is selected from group 1, 2, 3 or 4 as set forth in Table 1, above. Next, the subject inserts one odorous pod in each compartment of the receptacle. In certain embodiments, one odorous pod containing an odorant from one of group 1, 2, 3, or 4 are placed in sequential compartments.

In other embodiments, each compartment can contain an odorous pod that includes an odorant from the same group of odors. Here, the odors can be from the same category within a group and/or different categories from within the same group.

In certain embodiments the odorous chemicals present in each odorant pod of the can be of the same concentration or different. In a specific embodiment a subject is provided a plurality of odorous pods containing odorous chemicals from the same group of odors, whereby each odorous pod contains a different concentration of odorant. Here, an odorous pod containing a different concentration of an odorant is inserted in each compartment of a receptacle. In some embodiments the pods can be inserted that they are in sequential order such that the highest concentration is placed in compartment one and the odorous pod containing the lowest odorant concentration is inserted in the last compartment (i.e., descending), or vice versa, i.e., ascending in concentration.

In other embodiments, the user inserts an odorous pod from a different group of odorants into each of compartment. Here, each pod in a group of pods contains a different odorant, such that at least one pod in a group of pods contains at least one odorant from group 1, 2, 3 or 4, and the odorant(s) in each pod is selected from a different group than the odorant(s) in the other pods. In a preferred embodiment, a user will be provided with at least one odorous pod from each of the four groups of odorants. For example, a first odorous pod containing an odorous chemical or more than one odorous chemical from group 1 is inserted in one compartment, while a second odorous pod containing an odorous chemical or more than one odorous chemical from group 2, 3 or 4 of Table 1 is placed in a different compartment. Subsequently, a third odorous pod containing an odorous chemical from a different group than either of the first two odorous pods is inserted in a third compartment. This insertion step continues until each and every compartment is occupied by an odorous pod containing an odorous chemical from a different group of odors.

In an embodiment of the present disclosure, the methods include exposing a subject to each odorant in the odorous pods located in the receptacle. In certain embodiments, the subject is exposed to each odorant by activating the odorous pod (e.g., opening the compartment cover or removing a sealant layer). In specific embodiments a subject is exposed to an odorant or series of odorous chemicals by inhaling each exposed odorant through the subject's nose at least once. In other embodiments, the exposure and inhalation process of the present methods can be repeated more than once. In another embodiment, the exposure and inhalation process can be repeated at least 1 to 3 times per day, or more. In yet another embodiment, the exposure and inhalation process can be repeated daily for at least one week. In other embodiments, the daily exposure and inhalation process can be repeated for at least one month. In yet another embodiment the exposure and inhalation process of the present method is repeated by a subject for between 1 month and 60 months, inclusive.

In an embodiment in which the present methods are used in order to diagnose or evaluate a subject's olfactory function the exposure and inhalation process can occur at least once or can be repeated serially over a short period of time, e.g., 2 minutes to 60 minutes, about 1 to 10 minutes, about 2 to 9 minutes, about 3 to 8 minutes, about 4 to 8 minutes, or about 5 minutes.

In an embodiment, in which the present method is used for promoting, supporting, or enhancing olfactory function or in order to treat a subject with hyposmia or anosmia, the exposure and inhalation process is repeated more than once, over an extended period of time, e.g, one week, one month, multiple years, one year, or multiple years. More specifically, in an embodiment in which olfactory function is evaluated or a subject is diagnosed with hyposmia or anosmia, the subject may be challenged to identify one or more odors present the odorous pods during or after the exposure and inhalation process. Here, the exposed odorant arising from an odorous pod is inhaled deeply through the nose for about 1-5 seconds, for about 1-4 seconds, for about 1-3 seconds, for about 1-2 seconds, for about 2-3 seconds, for about 2-4 seconds, for about 1 second, 2 seconds, 3 seconds, 4 seconds, 5 seconds or more. Once the subject has inhaled an odorant the odorant is expelled from the olfactory system by exhaling. The subject may repeat this exposure, inhalation and exhalation process at least one time or more, while trying to identify an odor or odors emitted by the odorous pod(s). In certain embodiments, the user may be provided a key or legend describing the specific odorants present in each odorous pod.

In an embodiment where the odorous pods include odorants of varying concentrations, the exposed odorant arising from an odorous pod is inhaled deeply through the nose for about 1-5 seconds, for about 1-4 seconds, for about 1-3 seconds, for about 1-2 seconds, for about 2-3 seconds, for about 2-4 seconds, for about 1 second, 2 seconds, 3 seconds, 4 seconds, 5 seconds or more. Once the subject has inhaled an odorant, the odorant is expelled from the olfactory system by exhaling. The subject may repeat this exposure, inhalation and exhalation process at least one time or more, for each odorous pod until the odorant or odorants have been exhausted, e.g., is no longer detectable.

In certain embodiments, a receptacle with at least one compartment is provided, and each compartment houses a different odorous pod. In a specific embodiment, there are two, three, four, five, six, seven, eight, nine, ten, eleven, or twelve compartments that include odorous pods. In certain embodiments, after completion of an exposure, inhalation and exhalation process of the present disclosure an exhausted odorous pod is removed from a compartment, and a new odorous pod inserted into the now vacant compartment. The exposure, inhalation and exhalation process is then repeated with the replacement pods, as set forth above.

The entire process can take about 1 to 10 minutes, about 2 to 9 minutes, about 3 to 8 minutes, about 4 to 8 minutes, or about 5 minutes, and may be repeated one or more times. As stated above, the exposure and inhalation process of the present methods can be repeated at least 1 to 3 times per day, or more. In yet another embodiment exposure and inhalation process can be repeated daily for at least one week. In other embodiments, the daily exposure and inhalation process can be repeated for at least one month. In yet another embodiment the exposure and inhalation process of the present method is repeated by a subject for between 1 month and 60 months, inclusive, from about 6 months to about 48 months, from about 12 months to about 24 months, inclusive or for about 12 months.

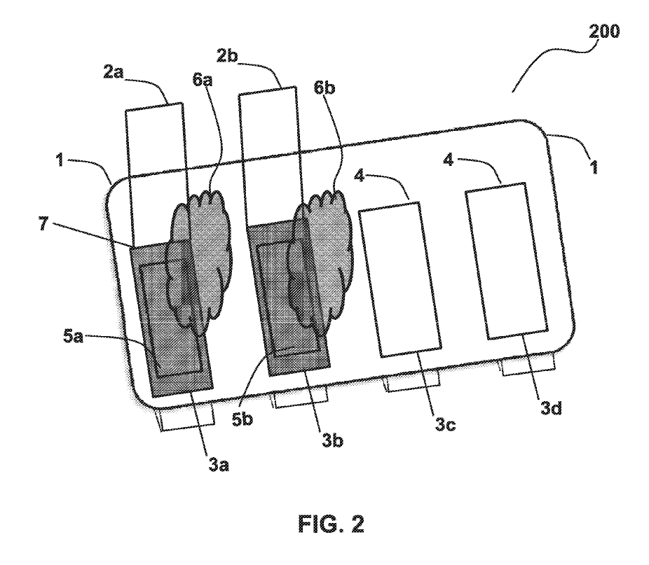

FIG. 2 shows a specific embodiment of the present disclosure including a receptacle 200 component of a kit of the present disclosure comprising four compartments 3, whereby each compartment includes an odorous pod 5 (shading). Here, the first 3a and second compartments 3b are in an open configuration after the odorous pod 5a present in the first compartment 3a was activated by the user by opening the lid 2a, exposing the odorant 6a to the external environment such that a scent is emitted for inhalation according to the present methods. Once the user inhales the emitted odorant 6a from the first compartment 3a, the user will then activate the odorous pod 5b present in the second compartment 3b by, for example, opening the lid 2b of the second compartment 3b and exposing the second odorant 6b to the external environment such that a second scent is emitted for inhalation. The remaining compartments 3c and 3d are shown in a closed configuration (e.g., sealed) 4 prior to activation by the user. Each compartment 3 contains an odorous pod 5 that, when released, emits an odorant 6 to be exposed and inhaled by a subject.

The foregoing methods are useful to promote, support, or enhance olfactory function and to treat hyposmia or anosmia in a subject in need thereof. The methods of the present disclosure are also useful to stimulate the mind of a subject in need thereof, including stimulating executive function, and memory. Given the well-established link between olfactory and cognitive functioning, repeated use of this device may be used to help stimulate and support the proper functioning of portions of the brain connected to the olfactory system (including the frontal, temporal, parietal, occipital lobes, cerebellum and brainstem), which function abnormally in conditions such as cognitive impairment, Alzheimer's disease and Parkinson's disease.

The present description, embodiments and examples provided therein should not be construed as limiting in any way. The contents of all cited references (including literature references, issued patents, and published patent applications as cited throughout this application) are hereby expressly incorporated by reference.

What is claimed is:

1. A kit for olfactory stimulation comprising;
a. a receptacle comprising at least four compartments for housing an odorous pod;
b. at least four odorous pods, wherein the odorant for each odorous pod is selected from Group 1, 2, 3 or 4 set forth in Table 1, and wherein the odorants of said at least four odorant pods are different from each other.

2. The kit of claim 1, further comprising instructions, wherein said instructions provide a user with methods of using said receptacle to promote, support or enhance olfactory function of the user.

3. The kit of claim 1, wherein at least one of said odorants of said at least four odorous pods is from Group 1, 2, 3 or 4 set forth in Table 1.

4. The kit of claim 1, wherein said receptacle comprises at least six compartments, and at least six odorous pods.

5. The kit of claim 4, wherein at least one of said odorants of said at least six odorous pods is from Group 1, 2, 3 or 4 set forth in Table 1.

6. The kit of claim 3, wherein the odorant pod containing an odorant from Group 1 comprises a spicy odorant chemical or an acid odorant chemical.

7. The kit of claim 3, wherein the odorant pod containing an odorant from Group 2 comprises a burned odorant chemical or an aromatic odorant chemical.

8. The kit of claim 3, wherein the odorant pod containing an odorant from Group 3 comprises a capryilic odorant chemical or a putrid odorant chemical.

9. The kit of claim 3, wherein the odorant pod containing an odorant from Group 4 comprises a fragrant odorant chemical, a fruity odorant chemical or a sweet odorant chemical.

10. The kit of claim 3, wherein said Group 1 odorant is an acid odorant or a spicy odorant, said Group 2 odorant is a burned odorant, said Group 3 odorant is a capryilic odorant or a putrid odorant, and said Group 4 odorant is a fragrant odorant, a fruity odorant or a sweet odorant.

11. The kit of claim 3, wherein said Group 1 odorant is an acid odorant, said Group 2 odorant is a burned odorant, said Group 3 odorant is a capryilic odorant, and said Group 4 odorant is a fragrant odorant.

12. The kit of claim 5, wherein at least two of said at least six odorous pods contain an odorant from Group 3 and at least two of said at least six odorous pods contain an odorant from Group 4.

13. The kit of claim 12, wherein said odorous pod containing a Group 1 odorant comprises an spicy odorant chemical, said odorous pod containing a Group 2 odorant comprises a burned odorant chemical, said odorous pods containing a Group 3 odorant include an odorous pod comprising a capryilic odorant chemical and another odorous pod comprising a putrid odorant chemical, and said odorous pods containing a Group 4 odorant include an odorous pod comprising a sweet odorant chemical, and another odorous pod comprising a fruity odorant chemical.

14. A method to promote, support, or enhance olfactory function in a subject comprising:
a. Providing a receptacle, comprising at least four compartments for housing an odorous pod;
b. Providing at least four odorous pods, wherein the odorant for each odorous pod is selected from Group 1, 2, 3 or 4 as set forth in Table 1, and wherein the odorants of said at least four odorant pods are different from each other;
c. Inserting one of said at least four odorous pods in each of said at least four compartments of said receptacle; and
d. Sequentially exposing a subject to each odorant, one after the other, until said subject has been exposed to each odorant present in said receptacle.

15. The method of claim 14, wherein at least one of said odorants of said at least four odorous pods is from Group 1, 2, 3 or 4 as set forth in Table 1.

16. The method of claim 14, wherein said receptacle comprises at least six compartments, and at least six odorous pods, and said at least one of said odorants of said at least six odorous pods is from Group 1, 2, 3 or 4 as set forth in Table 1.

17. The method of claim 14, wherein said exposing step comprises separately inhaling the odorant of each of said at least four odorant pods through said subject's nose.

18. The method of claim 14, further comprising repeating said exposing step at least 1 to 3 times in a day.

19. The method of claim 18, further comprising repeating steps a-d daily for at least one week.

20. The method of claim 19, further comprising repeating steps a-d daily for at least one month.

21. The method of claim 20, further comprising repeating steps a-d daily for a period of between 1 month and sixty months, inclusive.
